(19) United States
(12) Patent Application Publication
(10) Pub. No.: US 2014/0288632 A1
(43) Pub. Date: Sep. 25, 2014

(54) Graft Device with Adhered Fiber Matrix

(71) Applicant: Neograft Technologies, Inc., Taunton, MA (US)

(72) Inventors: Lorenzo Soletti, Pittsburgh, PA (US); Jon McGrath, Duxbury, MA (US); Mohammed S. El-Kurdi, Pittsburgh, PA (US); J. Christopher Flaherty, Auburndale, FL (US)

(21) Appl. No.: 14/354,025

(22) PCT Filed: Oct. 25, 2012
(86) PCT No.: PCT/US2012/061790

§ 371 Date: Apr. 24, 2014

Related U.S. Application Data
(60) Provisional application No. 61/551,249, filed on Oct. 25, 2011.

(51) Int. Cl. A61F 2/07

(52) U.S. Cl. CPC A61F 2/07
USPC 623/1.13

(57) ABSTRACT

A graft device including a tubular member and a fiber matrix surrounding the tubular member for a mammalian patient is disclosed. At least a portion of the fiber matrix is adhered to the tubular member. Methods and systems used in creating a graft device are also provided.

### RELATED APPLICATIONS

This application is a National Phase of International Application No. PCT/US2012/061790 filed Oct. 25, 2012, which claims benefit of priority to U.S. Provisional Application No. 61/551,249 filed Oct. 25, 2011, the contents of which are hereby incorporated herein by reference in their entirety.

This application is related to International Patent Publication Number WO/2008/094971, which claims priority to U.S. patent application Ser. No. 12/022,430, filed Jan. 30, 2008; International Patent Publication Number WO/2011/056705, filed Oct. 28, 2010; International Patent Publication Number WO/2011/084559, filed Dec. 16, 2010; and International Patent Publication Number WO/2012/097229, which claims priority to U.S. Provisional Application Ser. No. 61/432,914, filed Jan. 14, 2011; the contents of each of which is incorporated herein by reference in their entirety.

### FIELD OF THE INVENTION

The present invention relates generally to graft devices for mammalian patients including adhered layers, as well as systems and methods for creating graft devices with adhered layers.

### BACKGROUND OF THE INVENTION

Coronary artery disease, leading to myocardial infarction and ischemia, is a leading cause of morbidity and mortality worldwide. Current treatment alternatives include percutaneous transluminal angioplasty, stenting, and coronary artery bypass grafting (CABG). CABG can be carried out using either arterial or venous conduits and is the most effective and most widely used treatment to combat coronary arterial stenosis, with nearly 500,000 procedures being performed annually. In addition, there are approximately 80,000 lower extremity bypass surgeries performed annually. The venous conduit used for bypass procedures is most frequently the autogenous saphenous vein and remains the graft of choice for 95% of surgeons performing these bypass procedures. According to the American Heart Association, in 2004 there were 427,000 bypass procedures performed in 249,000 patients. The long term outcome of these procedures is limited due to occlusion of the graft vein or anastomotic site as a result of intimal hyperplasia (IH), which can occur over a timeframe of months to years.

Development of successful small diameter synthetic or tissue engineered vascular grafts has yet to be accomplished and use of arterial grafts (internal mammary, radial, or gastroepiploic arteries, for example) is limited by the short size, small diameter and availability of these veins. Despite their wide use, failure of arterial vein grafts (AVGs) remains a major problem: 12% to 27% of AVGs become occluded in the first year with a subsequent annual occlusive rate of 2% to 4%. Patients with failed AVGs usually require clinical intervention such as an additional surgery.

IH accounts for 20% to 40% of all AVG failures within the first 5 years after CABG surgery. Several studies have determined that IH develops, to some extent, in all mature AVGs and this development is regarded by many as an unavoidable response of the vein to grafting. IH is characterized by phenotypic modulation, followed by de-adhesion and migration of medial and adventitial smooth muscle cells (SMCs) and myofibroblasts into the intima where they proliferate. In many cases, this response can lead to stenosis and diminished blood flow through the graft. It is thought that IH may be initiated by the abrupt exposure of the veins to the dynamic mechanical environment of the arterial circulation.

### SUMMARY

For these and other reasons, there is a need for systems, methods and devices which provide enhanced AVGs and other improved grafts for mammalian patients. Desirably, the systems, methods and devices are expected to improve long term patency and minimize surgical and device complications.

Embodiments of the present inventive concepts can be directed to graft devices for mammalian patients, as well as systems and methods for creating these graft devices.

According to an aspect, a graft device for a mammalian patient comprises a tubular member and a fiber matrix surrounding the tubular member. At least a portion of the fiber matrix is adhered to the tubular member, such as to prevent or resist motion between the tubular member and the fiber matrix at one or more locations. The fiber matrix can restrict expansion of the tubular member. The tubular member can comprise a varying circumferential shape and the fiber matrix can conform to this varying circumferential shape. The fiber matrix can be bioerodible, non-bioerodible, or can include both bioerodible and non-bioerodible portions.

The fiber matrix can comprise one or more polymers, such as a fiber matrix comprising a thermoplastic co-polymer.

The fiber matrix can comprise multiple layers, including at least an inner layer and an outer layer. An inner layer can be applied with a wetness that causes adherence to the tubular member. Inner layer fibers can be configured to adhere to the tubular member Inner layer materials can include one or more adhesives, such as fibrin glue, to cause adherence of at least a portion of the fiber matrix inner layer to the tubular member. The inner layer can transition over time, such as to dry over a time period less than 2 hours or to have adhesive properties that deactivate over a time period of at least one day. The inner layer can include one or more additives, such as an additive selected from the group consisting of: microbeads; adhesive microbeads; mechanically adhesive particles; a glue-like substance such as fibrin glue; sprinkled tubules; reactive ligands; activated monomers or oligomers; fibrilar projections; catalytic agents; nucleating agents; reinforcing elements; coloration agents; and combinations of these.

At least a portion of the fiber matrix can be configured to bond with an associated portion of the tubular member. The fiber matrix can be adhered to the tubular member with a bond, such as a bond selected from the group consisting of: a permanent bond; a temporary bond; a superficial bond; a thrombus bond; a chemical cross link bond; an ionic bond; a covalent bond; a hydrogen bond; a mechanical bond; and combinations of these.

At least a portion of the fiber matrix can be attached to the tubular member with an adhesive. The adhesive can be applied to the entire length and/or circumference of the tubular member, or to discrete portions. In some embodiments, one or both ends of the tubular member is adhered to the fiber matrix, leaving the middle portion of the graft device void of adherence such that the fiber matrix can move relative to the tubular member in this middle portion. In other embodiments, the middle portion of the tubular member is adhered to the fiber matrix while one or both ends are void of adherence between the fiber matrix and the tubular member.

Adherence between the tubular member and the fiber matrix can be temporary or relatively permanent. Temporary adhesion can last for a period of time greater than one hour, greater than one week, greater than one month or greater than six months.

The graft device can comprise an adhesive configured to adhere the fiber matrix to the tubular member. In some embodiments, the adhesive comprises fibrin glue. Alternatively or additionally, the adhesive can comprise one or more components of the patient's blood, or components of blood from a donor patient. The graft device can include an adhesive selected from the group consisting of: fibrin glue; polyurethane; polyacrylamide; polyimide; epoxy; silicone; cyanoacrylate; polyacrylate; polyacrylamide; polyethylene oxide; poloxymer; a polysaccharide; a polymerized protein; a carbohydrate; a glycoprotein; a mucopolysaccharide; a polyphenolic protein; a reactive adhesive; a non-reactive adhesive; and combinations of these. The adhesive can comprise an adhesive that is bioerodible, hydrophilic and/or hydrophobic, or can include discrete portions that include these properties.

Adhesive can be applied to the tubular member in discrete locations, such as drops placed uniformly or randomly along the tubular member and/or a bead of adhesive placed in a helix along the tubular member. Adhesive can be applied to less than 10% of the tubular member surface, to a surface area between 10% and 30%, or to an area greater than 50% of the tubular member surface.

The graft device can include an adhesive layer positioned between the tubular member and the fiber matrix. The adhesive layer can comprise one or more portions, such as axial or longitudinal portions. The adhesive layer can comprise one or more layers, such as an inner layer and an outer layer. The adhesive layer can include a second fiber matrix, such as a restrictive fiber matrix similar or dissimilar to the first fiber matrix of the graft device. The adhesive layer can comprise a helical geometry. The adhesive layer can comprise a uniform or non-uniform thickness.

The graft device can include a temporary adhesive, such as an adhesive that can be deactivated during a surgery implanting the graft device. Deactivation can be accomplished via the introduction of one or more of: water; thermal energy; light; and combinations of these.

The tubular member can comprise living tissue, such as living tissue selected from the group consisting of: vein such as a saphenous vein; artery; urethra; intestine; esophagus; ureter; trachea; bronchi; duct; fallopian tube; and combinations of these. Alternatively or additionally, the tubular member can comprise non-living tissue material, such as non living tissue material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester based polymer; polyether based polymer; thermoplastic rubber; and combinations of these.

The tubular member can comprise an outer surface wherein at least a portion of the outer surface is treated to bond or otherwise adhere to at least a portion of the fiber matrix. Surface treatments include but are not limited to: surface energy modifications; chemical modifications; mechanical modifications; plasma discharge modifications; and combinations of these. Surface treatments can be configured to enhance the adherence between the tubular member and an adhesive, such as an adhesive layer.

In another aspect, a system for creating a graft device for a mammalian patient comprises a tubular member and a fiber matrix delivery assembly constructed and arranged to deliver a fiber matrix to the tubular member. At least a portion of the fiber matrix is adhered to the tubular member. The system can further comprise an adhesive applicator, which can be integral to the fiber matrix delivery assembly or a separate component. The adhesive applicator can deliver adhesive over the full length and/or the full circumference of the tubular member. The adhesive applicator can deliver adhesive to some portions of the tubular member but not others, such as to one or more ends of the tubular member while avoiding a middle segment. The adhesive applicator can deliver adhesive in a uniform or non-uniform thickness. The adhesive applicator can deliver a temporary adhesive, and it can deliver multiple adhesives.

The system can further comprise a surface modifier constructed and arranged to modify at least a portion of the tubular member. The surface modifier can be configured to provide a modification selected from the group consisting of: a surface energy modification; a chemical modification; a mechanical modification; and combinations of these.

The fiber matrix delivery assembly can comprise an electrospinning unit, such as an electrospinning unit configured to deliver an inner and outer layer of fiber matrix. The fiber matrix delivery assembly can include a nozzle, such as a nozzle constructed and arranged to have a variable distance between the nozzle tip and the tubular member, such as during fiber matrix delivery and/or adhesive delivery. Nozzle position can be varied to achieve a desired wetness of fiber, such as a wetness configured to cause adherence of the fiber matrix to the tubular member.

In another aspect, a method for creating a graft device for a mammalian patient comprises placing a fiber matrix about a tubular member such that at least a portion of the fiber matrix is adhered to the tubular member. The fiber matrix can be applied to the tubular member such that one or more fibers adhere to the tubular member. The method can further comprise applying an adhesive, such as an adhesive applied between the tubular member and the fiber matrix. The adhesive can be applied directly to the tubular member, such as prior to application of the fiber matrix. The adhesive can be applied in one or more discrete segments, or to the entire length and/or circumference of the tubular member. The adhesive can be applied with a nozzle, such as the same nozzle that applies the fiber matrix. The adhesive can be applied simultaneous with the application of the fiber matrix. The fiber matrix can be applied in a first time period in which the adhesive is also applied, and in a second time period in which no adhesive is applied.

The method can comprise applying an inner layer of fibers and an outer layer of fibers, and the inner layer of fibers can comprise different properties than the outer layer of fibers. The inner layer of fibers can comprise a wetness such that the inner fibers adhere to the tubular member. The inner fibers can be applied with a nozzle placed closer to the tubular member than the distance at which the outer fibers are applied.

### BRIEF DESCRIPTION OF THE DRAWINGS

FIG. 1 is a side sectional view of an example graft device including an adhesive layer, in accordance with the present inventive concepts;

FIG. 2A is a side sectional view of an example graft device including a partial adhesive layer at each end, in accordance with the present inventive concepts;

FIG. 2B is a side sectional view of an example graft device including a partial adhesive layer at a mid-portion, in accordance with the present inventive concepts;

FIG. 3A is a perspective view of an example graft device including a layer comprising two longitudinal segments, in accordance with the present inventive concepts;

FIG. 3B is a perspective view of an example graft device including a layer comprising multiple axial portions, in accordance with the present inventive concepts;

FIG. 3C is a perspective view of an example graft device including a layer comprising multiple braids arranged in a helical orientation, in accordance with the present inventive concepts;

FIG. 4A is a perspective view of an example harvested graft, in accordance with the present inventive concepts;

FIG. 4B is a perspective view of the harvested graft of FIG. 4A, after application of adhesive drops and a helical adhesive bead; in accordance with the inventive concepts;

FIG. 4C is a perspective view of the harvested graft of FIG. 4B, after application of a fiber matrix, in accordance with the inventive concepts;

FIG. 5 is a perspective view of an embodiment of an apparatus for producing a graft device, in accordance with the present inventive concepts; and

FIG. 6 is a flow chart depicting an example method for producing a graft device, in accordance with the present inventive concepts.

### DETAILED DESCRIPTION OF THE DRAWINGS

Reference will now be made in detail to the present embodiments of the systems and methods described herein, examples of which are illustrated in the accompanying drawings. The same reference numbers are used throughout the drawings to refer to the same or like parts.

Provided herein are graft devices for implantation in a mammalian patient. The graft devices include a tubular member, such as harvested tissue such as a harvested vein graft, and a fiber matrix that surrounds the tubular member. One or more discrete portions of the fiber matrix, or the entire inner surface of the fiber matrix is adhered the tubular member, such as to limit (e.g., prevent) relative motion between the tubular member and the fiber matrix. For purposes of this application, adherence of the fiber matrix to the tubular member shall be synonymous with adherence of the tubular member to the fiber matrix.

The adherence between the tubular member and the fiber matrix typically provides one or more advantages selected from the group consisting of: creating ease in handling in the implantation procedure, for example, in the creation of an anastomosis; improving long term patency, such as in preventing undesired motion between the fiber matrix and the tubular member; and reducing device and other complications during the implantation procedure and afterward. The adherence can be permanent or temporary, and can include portions configured to provide permanent and/or temporary adherence between associated portions of the tubular member and the fiber matrix.

Adherence between the tubular member and the fiber matrix can be caused by modifying the inner layer of the fiber matrix, such as to function as an adhesive. Alternatively or additionally, an adhesive such as fibrin glue, blood (e.g. the patient's own blood or blood from a donor), or other adhesive can be placed between the tubular member and the fiber matrix. The adhesive can be placed on the tubular member, the fiber matrix or both. Alternatively or additionally, the outer surface of the tubular member can be treated, such a treatment configured to cause the fiber matrix to adhere to the tubular member.

Also provided herein are systems and methods for creating a graft device comprising a tubular member and a surrounding fiber matrix, wherein at least a portion of the fiber matrix is adhered to the tubular member. Systems can include electrospinning and other fiber applying systems. Application of one or more adhesives can be performed using the same or similar equipment, simultaneous or sequential with the fiber application, such as through the same or a different nozzle.

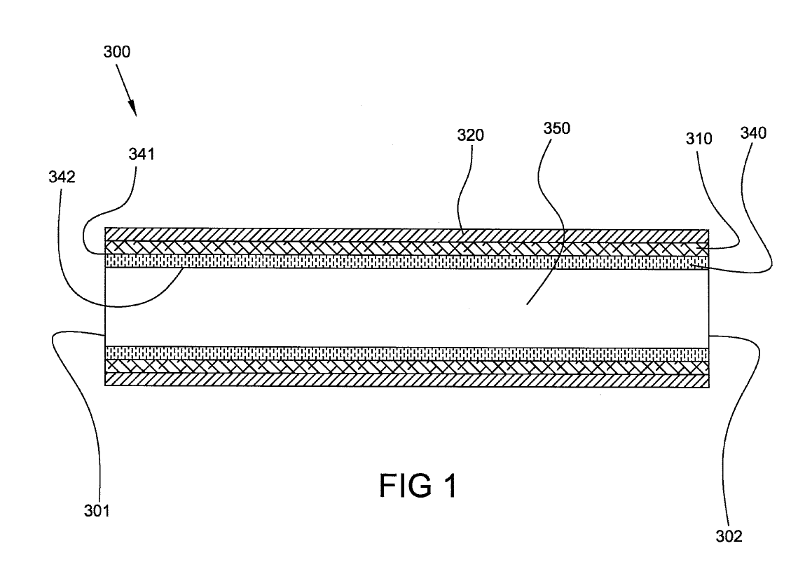

Referring now to FIG. 1, a side sectional view of a graft device including an adhesive layer is illustrated. Graft device 300 includes a tubular member, conduit 340, having inner wall 342 and outer wall 341, circumferentially surrounded by a covering, fiber matrix 320. At least a portion of fiber matrix 320 is adhesively attached to conduit 340. Graft device 300 includes a first end 301 and a second end 302, and can be configured to be placed between a first body location and a second body location of a patient. Graft device 300 includes lumen 350 from first end 301 to second end 302, such as to carry blood or other fluid when graft device 300 is connected between the two vessels, such as two blood vessels.

Adhesive layer 310 adheres outer wall 341 of conduit 340 to fiber matrix 320 across approximately the entire length and circumference of conduit 340. Layer 310 can be a portion of fiber matrix 320, such as an inner layer of matrix 320 configured to function as an adhesive, or it can be a separate layer or other separate component of graft device 300 configured to cause adherence of matrix 320 to tubular member 340. Adhesive layer 310 can be used to limit (e.g., prevent) undesired motion between conduit 340 and fiber matrix 320, such as during an anastomosis procedure in which relative motion between conduit 340 and fiber matrix 320 would complicate the attachment process. Adhesive layer 310 can include a material selected from the group consisting of: fibrin glue; polyurethane; polyacrylamide; polyimide; epoxy; silicone; cyanoacrylate; polyacrylate; polyacrylamide; polyethylene oxide; poloxymer; polysaccharides; polymerized proteins; carbohydrates; glycoproteins; mucopolysaccharides; polyphenolic proteins; reactive adhesives; non-reactive adhesives; and combinations of these. Alternatively or additionally, adhesive layer 310 can include blood, such as the patient's blood or components of the patient's blood. Alternatively, the blood can include a donor's blood or components of the donor's blood. Use of blood as an adhesive can provide additional benefits, such as improved biocompatibility, reduced irritation, reduced inflammation, and combinations of these.

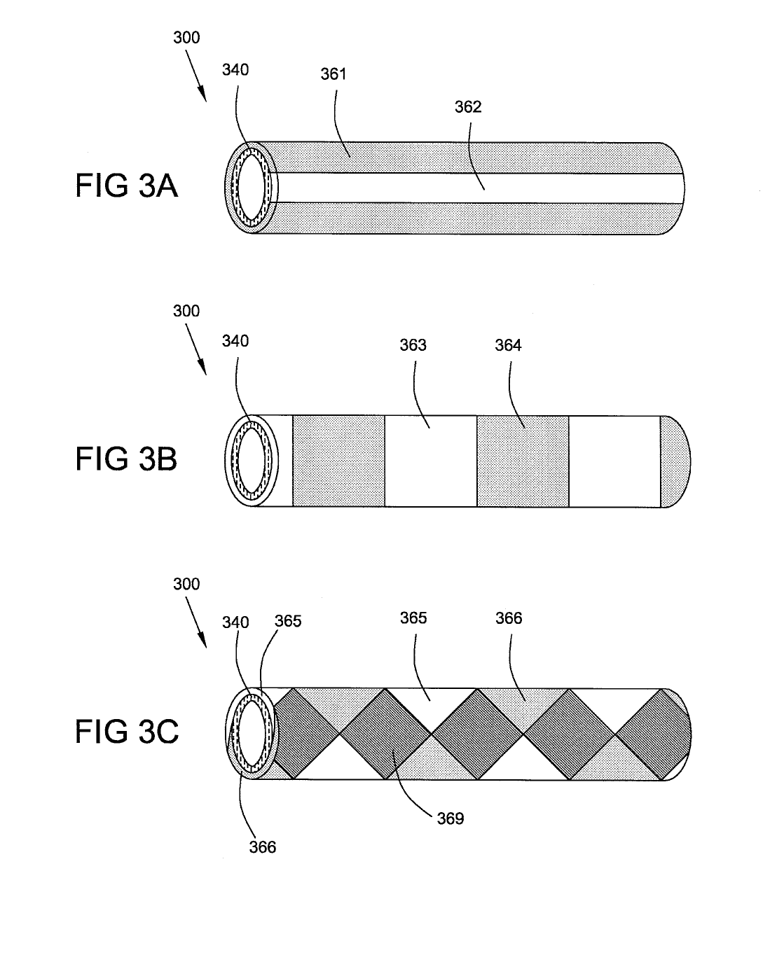

In some embodiments, adhesive layer 310 can include at least two portions, for example a first portion and a second portion, where the first portion and the second portion display different properties and/or can include different materials. In some embodiments, adhesive layer 310 and/or fiber matrix 320 can include at least two longitudinal segments, for example a first longitudinal segment and a second longitudinal segment, where the first and second segments display different properties and/or can include different materials, such as one or more adhesive materials, as described in reference to FIG. 3A herebelow. Alternatively, adhesive layer 310 can include at least two axial segments, for example a first axial segment and a second axial segment, where the first and second axial segments display different properties and/or can include different materials, such as one or more adhesive materials, as described in reference to FIG. 3B herebelow. Alternatively, adhesive layer 310 can comprise a braid of two or more materials, for example, where one or more of the braided materials include an adhesive, as described in reference to FIG. 3C herebelow.

Adhesive layer 310 can be selected and/or applied such that fiber matrix 320 temporarily adheres to conduit 340 for an amount of time ranging from less than one hour to less than six months, for example, less than one week or less than one month. Adhesive layer 310 can be applied such that at least a portion of fiber matrix 320 does not adhere to conduit 340. Adhesive layer 310 can display various properties, for example, adhesive layer 310 can be bioerodible, hydrophilic, and/or hydrophobic, or can include discrete portions that include these properties.

In some embodiments, adhesive layer 310 can be integrated into fiber matrix 320. For example, fiber matrix 320 can include multiple layers, e.g. an inner layer and an outer layer, such that fiber matrix 320 includes adhesive layer 310 and layer 310 is configured to adhere to conduit 340, potentially avoiding the need for a separate adhesive. Non-limiting examples of the types of bonding include: chemical bonds such as chemical cross link, ionic, covalent and hydrogen bonds; mechanical bonds; permanent bonds; temporary bonds; superficial bonds; thrombus bonds; and combinations of these.

In some embodiments, the inner layer of fiber matrix 320 can include materials similar to the outer layer of fiber matrix 320. Additionally or alternatively, the inner layer of fiber matrix 320 can display different physical properties than the outer layer. For example, the inner layer can include different wetness properties as compared to the outer layer, such as wetness properties created by controlling one or more properties of the fiber application process (e.g. a process parameter associated with solvent delivery and resultant solvent content of fiber matrix 320 at completion of deposition). In some embodiments, the inner layer can include a component at a particular concentration, and the outer layer can include a component at a different concentration. Alternatively or additionally, the components themselves can be the same or different across the layers. In some embodiments, the inner layer can include fibers with varying properties as compared to other fibers within the inner layer and/or as compared to the fibers included in the outer layer. For example, fibers can have a varying diameter across the inner layer to outer layers.

In some embodiments, the inner layer can include materials dissimilar to the materials of the outer layer of fiber matrix 320. For example, the inner layer can include an adhesive material, such as fibrin glue, where the outer layer does not include an adhesive. In some embodiments, the inner layer can be configured to transition over time, for example, the inner layer can dry over time, i.e. wetness decreasing over time. In some embodiments, the inner layer can include an additive agent. The agent can be in the form of micro-beads, adhesive micro-beads, mechanically adhesive particles, a glue-like substance such as fibrin glue, sprinkled tubules, reactive ligands, activated monomers or oligomers, fibrilar projections, catalytic agents, nucleating agents, reinforcing elements, coloration agents, and combinations of these. In some embodiments, the adhesive can be imageable and/or imaging-compatible, such as MRI imageable micro-beads that enable an image of one or more portions of graft device 300 to be generated post-implantation. For example, a clinician can desire to monitor the degradation of the graft device over time based upon the degradation of an imageable adhesive layer. In some embodiments, the adhesion created by adhesive and/or adhesive layer 310 can deactivate (e.g. an automatic or manual deactivation). Deactivation can occur at the time of implantation or at a time subsequent to the implantation (e.g. at least one day after implantation), such as when it is desirable for fiber matrix 320 to have a greater adhesion to conduit 340 during the creation and handling of graft device 300 as compared to post-implantation. For example, a water soluble adhesive can be included, and deactivation can occur upon the introduction of water. An adhesive layer including a poloxymer can be thermally deactivated. An adhesive layer including an ultraviolet-unstable component can be deactivated upon the introduction of light. Conversely, the adhesive and/or adhesive layer 310 can be configured activate at particular time, for example, delayed or post-implantation activation to maintain the placement of fiber matrix 320 with respect to conduit 340 and/or the surrounding tissue.

Fiber matrix 320 and/or adhesive layer 310 can be applied to conduit 340 via an electrospinning process, described in detail herebelow. Matrix 320 and adhesive layer 310 can be integrated into a single solution, or they can be maintained as separate solutions, such as two or more solutions applied simultaneously and/or sequentially.

In some embodiments, outer wall 341 of conduit 340 can be modified or otherwise treated such that bonding with fiber matrix 320 is enhanced. Similarly, outer wall 341 of conduit 340 can be treated such that bonding with adhesive layer 310 is enhanced. Non-limiting examples of conduit 340 surface treatments include: a surface energy modification; a chemical modification treatment; a mechanical modification treatment; a plasma discharge; and combinations of these.

Conduit 340 can include a portion having a varying cross-sectional shape, such that a fiber matrix 320 and/or adhesive layer 310 deposited on conduit 340 conforms to the varying circumferential shape over the portion of conduit 340. Fiber matrix 320 and/or adhesive layer 310 can have varying thicknesses, such as to accommodate a varying cross-sectional shape of conduit 340. Conduit 340 can include living tissue, for example, a vessel harvested from a mammalian patient. The conduit comprising living tissue can be selected from the group consisting of: vein, such as a saphenous vein; artery; urethra; intestine; esophagus; ureter; trachea; bronchi; duct; fallopian tube; and combinations of these. Alternatively or additionally, conduit 340 can be an artificial conduit such as a conduit constructed of materials selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester based polymer; polyether based polymer; thermoplastic rubber; and combinations of these.

In some embodiments, graft device 300 includes a restrictive matrix including polymers such as bioerodible polymers and/or non-bioerodible polymers. The fiber matrix can comprise a thermoplastic co-polymer made of two or more materials, such as a first material and a harder second material. In some cases, the co-polymer has a durometer of approximately 55 D, with approximately an even amount of the softer material and the harder material. The softer material can include polydimethylsiloxane (PDMS) and a polyether-based polyurethane. The harder material can include aromatic methylene diphenyl isocyanate (MDI).

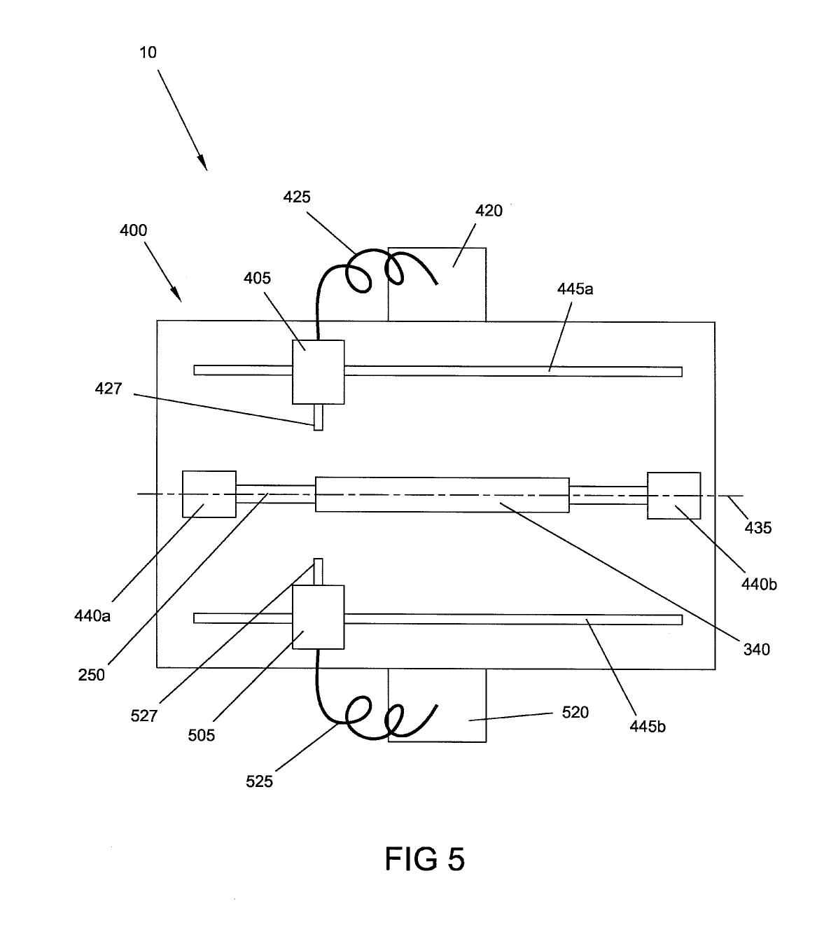

Graft device 300 can include a first portion, such as a first layer, that is bioerodible and a second portion, such as a second layer, that is not bioerodible. Fiber matrix 320 can be hydrophilic and/or contain one or more hydrophilic materials. Fiber matrix 320 can be applied using an electrospinning process, such as the electrospinning process described in detail herebelow in reference to FIG. 5. Further, an electrospinning process is disclosed in applicant's co-pending application, International Patent Publication Number WO/2012/097229, which claims priority to U.S. Provisional Application Ser. No. 61/432,914 filed Jan. 14, 2011, the contents of each of which are incorporated herein by reference in their entirety. The electrospinning process can be performed in an operating room, such as when conduit 340 is a harvested saphenous vein graft to be anastomosed between the aorta and a location on a diseased coronary artery distal to an occlusion. End to side anastomotic connections can be used to attach device 300 to the aorta and the diseased artery. Alternatively, a side to side anastomosis can be used, such as to attach an end of device 300 to multiple arteries in a serial fashion.

Fiber matrix 320 can be processed in a way specific to a patient morphological or functional parameter. These parameters can be selected from the group consisting of: vessel size such as diameter, length, and/or wall thickness; taper or other geometric property of a harvested vessel or other vessel intended for anastomotic attachment; size and location of one or more side branch ostium or antrum of the harvested vessel; patient age or sex; vessel elasticity or compliance; vessel vasculitis; vessel impedance; specific genetic factor or trait of the patient; and combinations of these. In embodiments where matrix 320 is deposited via electrospinning, conduit 340 can be free of any metal or magnetic material, such as metal clips used to ligate a side branch of a harvested saphenous vein.

Fiber matrix 320, when used for AVGs, can be processed in a way to achieve a certain blood flow rate or shear stress within the treated AVG. In some configurations, the shear stress achieved within the arterial vein graft is between 2-30 dynes/cm², such as between 12-20 dynes/cm². Fiber matrix 320 can be processed such that the oxygen, nutrients, or cellular permeabilities between the extravascular tissues and the abluminal surface of the treated hollow tissue is controlled. Such permeabilities can depend on a factor selected from the group consisting of: polymer or other fiber material chemical or physical property; fiber matrix pore size distribution; fiber matrix porosity; fiber matrix pore interconnectivity; and combinations of these. In a non-limiting example, cellular permeability can be selectively restricted to reduce leukocyte infiltration across the deposited fiber matrix with pore sizes smaller than seven microns and porosities between 50% and 95%. Generally, oxygen, nutrients, and cellular, e.g., endothelial cells, endothelial progenitor cells, etc. permeability are required to improve the treated hollow tissue in vivo remodeling and healing process. To this end, the pore size range is typically between 10 microns and 1000 microns, preferably between 200 microns and 500 microns, and the porosity typically ranges between 50% and 95%, preferably between 60% and 90%. The pores typically are highly interconnected so that a relatively straight path along the radial direction of fiber matrix 320 can be traced from most of the pores across the total thickness of matrix 320.

Radial constriction of saphenous vein grafts has been achieved with stent devices placed over the vein prior to anastomosing the graft to the targeted vessels. The devices of the present inventive concepts provide numerous advantages over the stent approaches. The devices of the present inventive concepts can have one or more parameters easily customized to a parameter of the harvested vessel and/or another patient parameter. The fiber matrix can be customized to a harvested vessel parameter such as vessel geometry, such as to reduce the vein internal diameter to produce desired flow characteristics. The fiber matrix can be customized to a target vessel parameter (e.g., the aorta and diseased artery), such as to be compatible with target vessel sizes and/or locations. The fiber matrix can be modified to simplify or otherwise improve the anastomotic connections, such as to be reinforced in the portion of the device that is anastomosed (e.g., portion where suture and/or clips pass through) and/or to protrude beyond the length of the tubular member and overlap other members connected to the graft device. The devices of the present inventive concepts can be made to a wide array of lengths during the fabrication procedure, without the need for cutting, converse to the cutting of a stent device, which might result in dangerously sharp edges. The fiber matrix is applied to the tubular member in a controlled, repeatable manner, by an apparatus such as an electrospinning instrument. The ends of the fiber matrix are atraumatic, avoiding tissue damage at the anastomotic sites. In addition, the fiber matrix of the present inventive concepts is easily and atraumatically removable, such as to apply another fiber matrix. Stent devices are applied manually by the clinician and suffer from numerous issues including but not limited to: requiring significant manipulation which could cause vessel trauma; limited reproducibility and accuracy; being difficult to reposition or remove, particularly without damaging the harvested vessel; and combinations of these.

The graft device disclosed herein can include any of various features described in applicant's co-pending U.S. Patent Publication Number US 2008/0208323, filed Jan. 30, 2008, and International Patent Publication Number WO/2011/084559, filed Dec. 16, 2010, the contents of which are hereby incorporated herein by reference in their entirety.

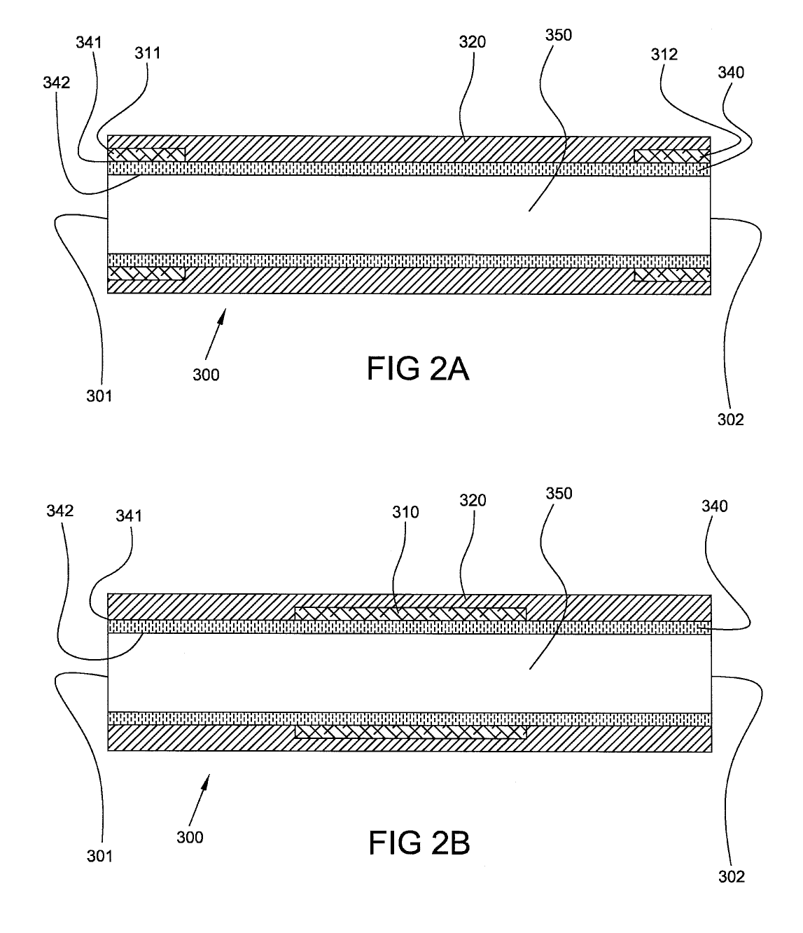

Referring now to FIGS. 2A and 2B, side sectional views of two graft devices including a partial adhesive layer are illustrated. Specifically, FIG. 2A includes graft device 300, similar to graft device 300 of FIG. 1, including a tubular member, conduit 340, having inner wall 342 and outer wall 341, circumferentially surrounded by a covering, fiber matrix 320. In the illustrated embodiment, adhesive layers 311 and 312 attach ends 301 and 302 of graft device 300, respectively, to fiber matrix 320. In this configuration, the mid portion of conduit 340 is allowed to move relative to fiber matrix 320 while device 300 portions proximate ends 301 and 302 remain substantially fixed, for example, to simplify creation of the anastomotic connections. Referring now to FIG. 2B, adhesive layer 310 attaches a mid portion of conduit 340 to fiber matrix 320, leaving the portions of tubular member 340 and fiber matrix 320 proximate ends 301 and 302, free to move relative to each other. Any portion or portions of conduit 340 can be attached to fiber matrix 320 via adhesive layer 310. Adhesive layers 310, 311 and 312 can comprise an inner layer of fiber matrix 320, such as an inner layer configured to function as an adhesive as has described in detail in reference to FIG. 1 hereabove. Adhesive layers 310, 311 and 312 can comprise a full circumferential tube (e.g. a 360° layer) or partial circumferential segments can be incorporated.

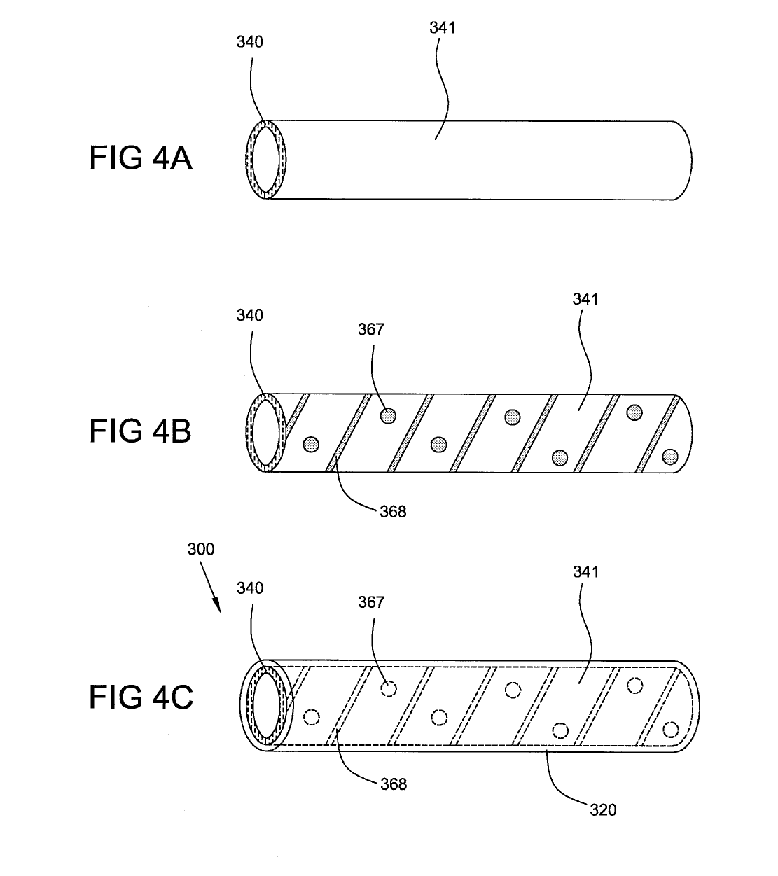

Referring now to FIGS. 3A-3C, perspective views of three graft devices, each including multiple segmented portions, are illustrated. In some embodiments, as illustrated in FIG. 3A, device 300 includes at least one layer having at least two longitudinal segments, for example, first longitudinal segment 361 and second longitudinal segment 362. First longitudinal segment 361 and second longitudinal segment 362 each surrounds a partial circumferential portion of conduit 340 and extend along the length of device 300. In some embodiments, first segment 361 and second segment 362 exhibit different properties and/or can include different materials. In some embodiments, first segment 361 includes an adhesive and second segment 362 does not include an adhesive. In this embodiment, segments 361 and 362 each can include a fiber matrix, similar to fiber matrix 320 discussed herein. For example, first segment 361 can include both an adhesive and a fiber matrix, and second segment 362 can include only the fiber matrix. To achieve this construction, an adhesive can be applied to conduit 340 along at least one longitudinal segment, for example longitudinal segment 361, and the fiber matrix can be electrospun onto the full circumference of conduit 340, as is described in reference to FIG. 4 herebelow.

In some embodiments, as illustrated in FIG. 3B, device 300 includes at least one layer having at least two axial segments, for example first axial segment 363 and second axial segment 364, each axial segment including a full circumferential layer surrounding a partial length of conduit 340. In some embodiments, first segment 363 and second segment 364 exhibit different properties and/or can include different materials, such as the presence of an adhesive and the lack thereof, similar to first segment 361 and second segment 362 described in reference to FIG. 3A hereabove. Alternatively, a first adhesive can be included in first segment 363, and a second, different adhesive can be used in second segment 364, such as a first adhesive which is relatively permanent and a second adhesive which is temporary.

In some embodiments, as illustrated in FIG. 3C, device 300 includes at least one layer having at least two segments that are helically oriented, for example, first segment 365 and second segment 366 which surround conduit 340. In some embodiments, first segment 365 and second segment 366 exhibit different properties and/or can include different materials, such as the presence of an adhesive and the lack thereof, similar to first segment 361 and second segment 362 described in reference to FIG. 3A hereabove. In some cases, segment 365 and segment 366 can be applied in a weave pattern such as an over-under helical braid, as illustrated in FIG. 3C, with overlap portions 369 positioned as shown. In some cases, segment 365 and 366 can be applied in a helical pattern along the length of conduit 340 such that overlap between segments 365 and 366 is reduced or eliminated.

Referring now to FIGS. 4A-4C, side perspective views of a three-step process for making a graft device are illustrated. FIG. 4A shows conduit 340 after a harvesting procedure has been performed as described in detail herein. Conduit 340 includes outer surface 341. FIG. 4B shows conduit 340 after multiple adhesive segments 367 have been applied. Adhesive segments 367 can comprise the circular patterns shown, or other geometries, and can be applied as a drop and/or with a nozzle or other applicator device. Alternatively or additionally, an adhesive segment 368 can be applied comprising one or more lines of adhesive, such as one or more helical beads of adhesive that are placed on conduit 340, such as adhesive dispensed through a translated nozzle delivering adhesive while conduit 340 is being rotated. Adhesive segments 367 and/or segments 368 can adhere to outer wall 341 of conduit 340. In some embodiments, adhesive segments 367 and segments 368 cover discrete portions of outer wall 341, with the total surface area of adhesive coverage limited to less than 30% and potentially less than 10% of the total surface area of wall 341. In some embodiments, surface area covered by segments 367 and/or segments 368 is more than 30% of the total surface area of wall 341, such as more than 50% of the surface area. Adhesive segments 367 and/or 368 can be applied in a random, scattered pattern, or they can be uniformly distributed about the length and circumference of conduit 340.

FIG. 4C shows a graft device 300 including fiber matrix 320 coving conduit 340 and adhesive segments 367 and 368, such that fiber matrix 320 is adhered to outer wall 341 of conduit 340. Fiber matrix 320 can be applied using an electrospinning, braiding, or other fiber application process, such as the electrospinning process described in reference to FIG. 5 herebelow.

Referring now to FIG. 5, a perspective view of an apparatus for producing a graft device is illustrated. Apparatus 10 includes electrospinning unit 400 and mandrel 250, where conduit 340 has been placed around mandrel 250. Conduit 340 can include living tissue and/or artificial materials, as is described herein. Electrospinning unit 400 can include one or more nozzle assemblies, and in the illustrated embodiment, unit 400 includes nozzle assembly 405 and 505, which includes nozzles 427 and 527, respectively. For clarification, any reference to a “nozzle” and “nozzle assembly” in singular or plural form can include one or more nozzles, such as nozzles 427 and 527 and one or more nozzle assemblies, such as nozzle assemblies 405 and 505. In one non-limiting embodiment, nozzle 427 can deliver a polymer solution while nozzle 527 can deliver an adhesive layer, such as the adhesive layer described in reference to FIG. 1. In an alternative embodiment, nozzle 427 delivers both polymer solution and adhesive, simultaneously or sequentially, avoiding the need for nozzle 527 or making nozzle 527 available for the delivery of another solution or compound, for example, a drug or an agent. Typically, the polymer solution includes one or more polymers and one or more solvents. Polymers can be selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of these. Solvents can be selected from the group consisting of: hexafluoroisopropanol; acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations of these.

In some embodiments, the fiber matrix can include an inner layer and an outer layer, where the inner layer can include an adhesive component and/or exhibit adhesive properties. The inner layer can be delivered separate from the outer layer, for example, delivered from a separate nozzle or at a separate time during the process. In some embodiments, electrospinning unit 400 can be configured to deliver the polymer solution and/or an adhesive layer according to set parameters. For example, an adhesive layer can be delivered to conduit 340 for a particular length of time, followed by delivery of a polymer solution for another particular length of time. Other typical application parameters include but are not limited to: amount of adhesive layer and/or polymer solution delivered; rate of adhesive layer and/or polymer solution delivered; nozzle distance to mandrel 250 and/or conduit 340; linear travel distance of a nozzle along its respective drive assembly (for example, drive assembly 445a and 445b); linear travel speed of a nozzle along its respective drive assembly; compositions of the polymer solution and/or adhesive layer; concentrations of the polymer solution and/or adhesive layer; solvent compositions and/or concentrations; fiber matrix inner and outer layer compositions and/or concentrations; spontaneous or sequential delivery of the polymer solution and the adhesive layer; voltage applied to the nozzle; voltage applied to the mandrel; viscosity of the polymer solution; temperature of the treatment environment; relative humidity of the treatment environment; airflow within the treatment environment; and combinations of these.

Nozzles 427 and 527 can be constructed of stainless steel. In one embodiment, nozzles 427 and/or 527 have a tubular construction with a length of approximately 1.5″, an ID of approximately 0.047″ and an OD of approximately 0.065″. Nozzles 427 and 527 can include an insulating coating, with the tip of nozzles 427 and/or 527 exposed (e.g. non-insulated), such as with an exposed length of approximately 1 cm. Nozzle geometry and electrical potential voltages applied between nozzles 427 and/or 527 and mandrel 250 are chosen to control fiber generation. In some embodiments, fibers are created with a diameter between 0.1 μm and 2.0 μm, such as with a diameter between 0.1 μm and 1.0 μm.

Mandrel 250 is positioned in a particular spaced relationship from nozzle assemblies 405 and/or 505 and nozzles 427 and/or 527, respectively. In some embodiments, as illustrated, mandrel 250 is positioned above and below nozzle assemblies 405 and 505. Alternatively, mandrel 250 can be positioned either above or below the nozzle assemblies 405 and/or 505. In some embodiments, mandrel 250 is located to the right or left of nozzle assemblies 405 and 505, or both left and right. The distance between mandrel 250 and the tip of nozzles 427 and 527 can be less than 20 cm, such as less than 15 cm. In a particular embodiment, the tip of nozzles 427 and/or 527 is approximately 12.5 cm from mandrel 250. As illustrated, multiple nozzles 427 and 527, for example nozzles of similar or dissimilar configurations, can be positioned in various orientations relative to mandrel 250. In some embodiments, the distance between nozzles 427 and/or 527 and mandrel 250 and/or conduit 340 varies such that the adhesive layer and polymer solution display various properties, for example, wetness of the adhesive layer and/or the fiber matrix layer. In some embodiments, nozzles 427 and/or 527 distance can vary continuously during the electrospinning process or can vary for a set period of time during the process.

An electrical potential can be applied between nozzles 427 and/or 527 and one or both of conduit 340 and mandrel 250. The electrical potential can draw at least one fiber from nozzle assemblies 405 and/or 505 to conduit 340. Conduit 340 can act as the substrate for the electrospinning process, collecting the fibers that are drawn from nozzle assemblies 405 and/or 505 by the electrical potential. In some embodiments, mandrel 250 and/or conduit 340 has a lower voltage than nozzles 427 and/or 527 to create the desired electrical potential. For example, the voltage of mandrel 250 and/or conduit 340 can be a negative or zero voltage while the voltage of nozzles 427 and/or 527 can be a positive voltage. Mandrel 250 and/or conduit 340 can have a voltage of about −5 kV (e.g., −10 kV, −9 kV, −8 kV, −7 kV, −6 kV, −5 kV, −4.5 kV-4 kV, −3.5 kV, −3.0 kV, −2.5 kV, −2 kV, −1.5 kV, −1 kV) and nozzles 427 and/or 527 can have a voltage of about +15 kV (e.g., 2.5 kV, 5 kV, 7.5 kV, 12 kV, 13.5 kV, 15 kV, 20 kV). In some embodiments, the potential difference between nozzles 427 and/or 527 and mandrel 250 and/or conduit 340 can be from about 5 kV to about 30 kV. This potential difference draws fibers from nozzles 427 and/or 527 to conduit 340. In some embodiments, nozzle 427 and/or 527 is placed at a potential of +15 kV while mandrel 250 is placed at a potential of −5 kV. In some embodiments, mandrel 250 is a fluid mandrel, such as the fluid mandrel described in applicant's co-pending International Patent Publication Number WO/2012/092138, which claims priority to U.S. Provisional Application Ser. No. 61/427,993 filed on Dec. 29, 2010, the contents of each of which are incorporated by reference herein in their entirety.

In some embodiments, a polymer solution, stored in polymer solution dispenser 420, can be delivered to nozzle assembly 405 through a polymer solution delivery tube 425. The electrical potential between nozzle 427 and conduit 340 and/or mandrel 250 can draw the polymer solution through nozzle 427 of nozzle assembly 405. Electrostatic repulsion, caused by the fluid becoming charged from the electrical potential, counteracts the surface tension of a stream of the polymer solution at nozzle 427 of the nozzle assembly 405. After the stream of polymer solution is stretched to its critical point, one or more streams of polymer solution emerges from nozzle 427 of nozzle assembly 405, and/or at a location below nozzle assembly 405, and move toward the negatively charged conduit 340. Using a volatile solvent, the solution dries substantially during transit and the fiber is deposited on conduit 340. Similarly, a solution comprising an adhesive, stored in adhesive solution dispenser 520, can be delivered to nozzle assembly 505 through adhesive solution delivery tube 525 via electrostatic repulsion. In some embodiments, adhesive solution can be delivered via assembly 405, and polymer solution can be delivered via assembly 505. Alternatively or additionally, an adhesive is provided in dispenser 420, such that it can be delivered through nozzle assembly 427. Delivery of the adhesive can be simultaneous with polymer fiber delivery (e.g. when the adhesive is mixed with polymer solution in dispenser 420), or sequential with polymer fiber delivery (e.g. when the adhesive is provided separate from the polymer solution in dispenser 420).

Mandrel 250 is configured to rotate about an axis, such as axis 435, with nozzles 427 and/or 527 perpendicular to axis 435. The rotation around axis 435 allows the fiber matrix to be deposited along all sides, or around the entire circumference of conduit 340. Mandrel 250 can be rotated by at least one motor 440a, 440b in direct or indirect communication with the ends of mandrel 250. In some embodiments, electrospinning unit 400 includes a single motor that rotates one end of mandrel 250. In some embodiments, two motors 440a and 440b are used. For example, motor 440a can be in communication with one end of mandrel 250 while motor 440b is in communication with the opposite end of mandrel 250. The rate of rotation of mandrel 250 can depend on how the fiber matrix is to be applied to conduit 340. For example, for a thicker fiber matrix, the rotation rate can be slower than if a thinner fiber matrix is desired.

In addition to mandrel 250 rotating around axis 435, the nozzle assembly 405 can move, such as when driven by drive assembly 445a in a reciprocating or oscillating horizontal motion. Drive assemblies 445a and/or 445b comprise a linear drive assembly, not shown, but typically a belt driven drive assembly comprising two or more pulleys driven by one or more stepper motors. Additionally or alternatively, nozzle assemblies 405 and/or 505 can be constructed and arranged to rotate around axis 435, rotating means not shown. The length of drive assemblies 445a and/or 445b and the linear motion applied to nozzle assemblies 405 and 505, respectively, can vary based on the length of conduit 340 to which a fiber matrix and/or adhesive layer will be delivered. For example, the supported linear motion of drive assemblies 445a and/or 445b can be about 10 cm to about 50 cm. Nozzle assemblies 405 and/or 505 can move along drive assemblies 445a and/or 445b, respectively, to apply a fiber matrix and/or adhesive layer to the entire length, or specific portions of a length, of conduit 340. In some embodiments, fiber(s) and/or adhesive is applied to the entire length of conduit 340 plus an additional 5 cm (to mandrel 250) on either end of conduit 340. In another embodiment, fiber(s) and/or adhesive is applied to the entire length of conduit 340 plus at least 1 cm beyond either end of conduit 340.

Nozzle assemblies 405 and/or 505 can be controlled such that specific portions along the length of conduit 340 are reinforced with a greater amount of fiber matrix as compared to other or remaining portions. In addition, conduit 340 can be rotating around axis 435 while nozzle assemblies 405 and/or 505 is moving along drive assemblies 445a and/or 445b, respectively, to provide control over the location on conduit 340 where the fiber matrix will be applied. In some embodiments, nozzle assemblies 405 and/or 505 are translated back and forth at a velocity of approximately 200 mm/sec. Rotational speeds of mandrel 250 and translational speeds of nozzle assemblies 405 and/or 505 can be relatively constant, or can be variable during the process.

Apparatus 10 can also include a power supply, not shown, but configured to provide the electric potentials to nozzles 427 and/or 527 and mandrel 250, as well as supply power to other components of apparatus 10 such as drive assemblies 445a and 445b. The power supply can be connected, either directly or indirectly, to at least one of mandrel 250 and conduit 340. Power can be transferred from the power supply to mandrel 250 and/or conduit 340 by, for example, a wire.

Apparatus 10 can also include inlet and/or outlet ports, not shown, but typically configured to control the environment surrounding nozzles 427 and/or 527 and/or the environment surrounding mandrel 250. A port can be configured to be both an inlet port and an outlet port. Apparatus 10 can include a housing, also not shown, but typically attachable to electrospinning unit 400 and defining a chamber surrounding nozzles 427 and/or 527 and/or mandrel 250, such that the ports can control a more limited (smaller) environment surrounding nozzles 427 and/or 527 and/or mandrel 250. Additionally or alternatively, the ports can be used to introduce or remove one or more gases, introduce or remove humidity, control temperature, control sterility, provide other environmental controls, and combinations of these.

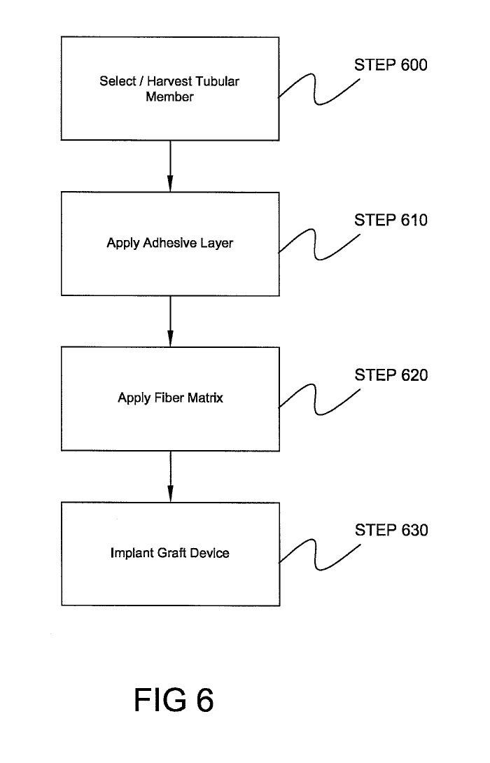

Referring now to FIG. 6, a method for creating a graft device is depicted. A conduit is selected and harvested from a patient, an adhesive layer and fiber matrix is applied to the conduit, and the resulting graft device is implanted into a patient.

In STEP 600, a conduit is selected for future implantation into a patient. The conduit can include living and/or artificial tissues, as are described in reference to FIG. 1 hereabove. In the illustrated method, the conduit includes living tissue, for example, a vessel to be harvested from a mammalian being, such as the patient. In the case of living tissue, the conduit can be selected based upon properties that would be desirable for use in a graft device, which can be subsequently used in a medical procedure, such as a bypass procedure. The particular segment (i.e. length) of conduit to be harvested can also be determined. Subsequent to selecting a conduit, the conduit is harvested, typically from the patient to receive the graft device, such as harvesting via surgical or minimally invasive surgical techniques known to those of skill in the art. Additional procedures and/or treatments can be performed, such as the ligation of one or more side branches present along the length of the selected conduit.

In STEP 610, an adhesive layer is applied to the harvested conduit. In some embodiments, the adhesive layer comprises one or more adhesives configured to adhere at least a portion of the fiber matrix to the tubular member, examples of which have been described hereabove.

In STEP 620, a fiber matrix is applied to the harvested conduit. The fiber matrix typically comprises one or more fiber matrices described herein, applied via one or more of the processes described herein. In some embodiments, STEPs 610 and 620 can be performed sequentially, in any order. In some other embodiments, STEPs 610 and 620 can be performed, at least in part, simultaneously, such as when the adhesive layer comprises the inner layer of the fiber matrix, as has been described in detail in reference to FIG. 1 hereabove. In some cases, a separate adhesive can be avoided, such as when adhesive forces are created by altering a property of the fiber matrix itself, such as the wetness of the fiber matrix. Alternatively, one or more adhesive components can be added to further adhere the fiber matrix to the tubular member.

The fiber matrix can comprise a polymer fiber matrix delivered with an apparatus such as the electrospinning unit described in reference to FIG. 5 hereabove. The fiber matrix can comprise fibers, such as fibers of varying or consistent properties. The fiber matrix can be configured to remain permanently implanted or to bioerode over time. In a particular embodiment, the fiber matrix can include both a bioerodible portion and a portion configured to remain stable over time (e.g. time periods of at least six months).

In STEP 630 the graft device is implanted in the patient during a medical procedure, for example, during an arterial bypass procedure. Specifically, the graft device can be anastomosed between the aorta and a location on a diseased coronary artery distal to an occlusion. Alternatively, a bypass procedure can be performed in the limb of the patient, such as in the patient's leg. End to side anastomotic connections can be used to attach the graft device to a source of oxygenated blood and a diseased artery. Alternatively, a side to side anastomosis can be used, such as to attach an end of the graft device to multiple arteries in a serial fashion.

While certain embodiments of the systems, methods and devices have been described in reference to the environment in which they were developed, they are merely illustrative of the principles of the inventions. Modification or combinations of the above-described assemblies, other embodiments, configurations, and methods for carrying out the inventive concepts described herein, and variations of aspects of the inventive concepts described herein that are obvious to those of skill in the art are intended to be within the scope of the claims. In addition, where this application has listed the steps of a method or procedure in a specific order, it can be possible, or even expedient in certain circumstances, to change the order in which some steps are performed, and it is intended that the particular steps of the method or procedure claim set forth herebelow not be construed as being order-specific unless such order specificity is expressly stated in the claim. Therefore, other embodiments are within the scope of the claims.

What is claimed is:

1-125. (canceled)

126. A graft device for a mammalian patient, the device comprising:
a tubular member; and
a fiber matrix surrounding the tubular member,
wherein at least a portion of the fiber matrix is adhered to the tubular member.

127. The device of claim 126 wherein the tubular member comprises a varying non-circular outer shape and wherein the fiber matrix comprises a deposition coating that conforms to a native shape of the varying non-circular outer shape of the tubular member.

128. The device of claim 126 wherein the fiber matrix comprises a polymer.

129. The device of claim 128 wherein the polymer comprises a thermoplastic co-polymer.

130. The device of claim 126 wherein the fiber matrix comprises at least an inner layer and an outer layer.

131. The device of claim 130 wherein the inner layer comprises similar materials to the outer layer.

132. The device of claim 130 wherein the inner layer includes an additive selected from the group consisting of: microbeads; adhesive microbeads; mechanically adhesive particles; a glue-like substance; a glue-like substance comprising fibrin glue; sprinkled tubules; reactive ligands; activated monomers or oligomers; fibrilar projections; catalytic agents; nucleating agents; reinforcing elements; coloration agents; and combinations thereof.

133. The device of claim 126 wherein approximately an entire length of the fiber matrix along the tubular member is adhered to the tubular member.

134. The device of claim 126 wherein the fiber matrix comprises a first end portion, a mid portion, and a second end portion and wherein at least the first end portion is adhered to the tubular member.

135. The device of claim 134 wherein at least the second portion is adhered to the tubular member.

136. The device of claim 135 wherein the mid portion is not adhered to the tubular member.

137. The device of claim 126 further comprising an adhesive wherein the at least a portion of the fiber matrix is adhered to the tubular member by the adhesive.

138. The device of claim 137 wherein the adhesive is selected from the group consisting of: fibrin glue; polyurethane; polyacrylamide; polyimide; epoxy; silicone; cyanoacrylate; polyacrylate; polyacrylamide; polyethylene oxide; poloxymer; a polysaccharide; a polymerized protein; a carbohydrate; a glycoprotein; a mucopolysaccharide; a polyphenolic protein; a reactive adhesive; a non-reactive adhesive; and combinations thereof.

139. The device of claim 137 wherein the adhesive comprises a bioerodible material.

140. The device of claim 126 wherein the tubular member comprises an outer surface comprising a surface treatment constructed and arranged to adhere the fiber matrix to the tubular member.

141. The device of claim 140 wherein the surface treatment comprises a surface energy modification treatment.

142. The device of claim 140 wherein the surface treatment comprises a chemical modification treatment.

143. The device of claim 140 wherein the surface treatment comprises a mechanical modification treatment.

144. The device of claim 140 wherein the surface treatment comprises a plasma discharge treatment.

145. The device of claim 126 wherein the tubular member comprises tissue selected from the group consisting of: vein such as a saphenous vein; artery; urethra; intestine; esophagus; ureter; trachea; bronchi; duct; fallopian tube; and combinations thereof.
